(19) United States
(12) Patent Application Publication
(10) Pub. No.: US 2016/0235776 A1
(43) Pub. Date: Aug. 18, 2016

(54) SUSTAINED-RELEASE COMPOSITIONS COMPRISING TOPIRAMATE AND AN ALKALIZER

(71) Applicant: CADILA HEALTHCARE LIMITED, Ahmedabad (IN)

(72) Inventors: Sushrut Krishnaji KULKARNI, Ahmedabad (IN); Ajaykumar HANDA, Ahmedabad (IN); Amit Om Prakash GUPTA, Ahmedabad (IN)

(73) Assignee: CADILA HEALTHCARE LIMITED, Ahmedabad (IN)

(21) Appl. No.: 15/019,094

(22) Filed: Feb. 9, 2016

(30) Foreign Application Priority Data
Feb. 13, 2015 (IN) 469/MUM/2015

(51) Int. Cl. A61K 31/7048; A61K 9/48; A61K 9/16

(52) U.S. Cl. CPC A61K 31/7048; A61K 9/167; A61K 9/4833; A61K 9/4808; A61K 9/1682

(57) ABSTRACT

The present invention relates to a sustained release composition comprising topiramate and one or more pharmaceutical excipients, wherein the composition comprises a single population of beads, wherein each bead comprises: a) an inert core, b) an inner layer comprising topiramate, c) an outer layer comprising at least one sustained release material and at least one alkalizer. It also relates to methods of preparing such compositions and using those compositions in the treatment of neurological and/or psychiatric condition.

### FIELD OF INVENTION

The present invention relates to a sustained release composition comprising topiramate and one or more pharmaceutical excipients, wherein the composition comprises a single population of beads, wherein each bead comprises: a) an inert core, b) an inner layer comprising topiramate, c) an outer layer comprising at least one sustained release material and at least one alkalizer. It also relates to methods of preparing such compositions and using those compositions in the treatment of neurological and/or psychiatric condition.

### BACKGROUND OF THE INVENTION

Topiramate is designated chemically as 2,3:4,5 Di-O-isopropylidene-β-D-fructopyranose sulfamate and has the following structural formula:

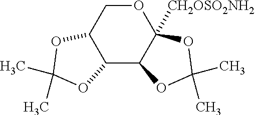

Topiramate is a sulfamate substituted monosaccharide which under the tradename TOPAMAX® has been approved for use as an antiepileptic agent, as an adjuvant therapy for patients with partial onset seizures or primary generalized tonic-clonic seizures, and for the prevention of migraine (U.S. Pat. No. 4,513,006).

For the treatment of epilepsy, the recommended dose of TOPAMAX® is 400 mg/day in one or multiple doses. For the treatment of epilepsy in adults, treatment is initiated with a dose of 25-50 mg/day, with the dose being titrated in increments of 25-50 mg at weekly intervals to the recommended or effective dose. Topamax® is an immediate release composition. Adverse effects associated with the administration of Topamax® include, but are not limited to, somnolence, dizziness, ataxia, speech disorders and related speech problems, psychomotor slowing, abnormal vision, difficulty with memory, paresthesia, diplopia, renal calculi (kidney stones), hepatic failure, pancreatitis, renal tubular acidosis, acute myopia and secondary angle closure glaucoma.

Though topiramate has a relatively long half-life of 21 hours in vivo, it usually results in severe side-effects that often result with peak plasma levels of the drug when taken in high doses. Thus, Topamax® is typically taken in multiple, “divided” doses, usually twice-daily (“BID”). However, administration of the medicament in this manner is cumbersome and patients can forget to take their medication in a timely manner. Moreover, each administration of a dose is associated with a peak in plasma concentrations of the drug, and the fluctuations associated with the peaks and valleys of blood plasma levels of the drug are undesirable. Therefore, there is a need for a composition of topiramate, which reduces or eliminates the side effects associated with peaking and fluctuating plasma levels of the drug and preferably may be administered in a once-daily regimen.

PCT application No. WO 2014-143380 discloses an extended-release topiramate capsule that includes a capsule shell containing a single population of coated particles; wherein each coated particle includes a core and a coating thereon; wherein each particle core includes a homogeneous mixture comprising topiramate throughout its core; and wherein the coating includes one or more release controlling agent(s). The specification discloses the use of a stabilizer in the core which can be used in an amount of at least 1% to at least 2% and up to 10% by weight of the core. Surprisingly, the present invention stabilizes topiramate in the composition efficiently with a low stabilizer concentration when used in the coating.

A form of topiramate has been described in PCT application No. WO 2008-061226 for once-daily sustained-release dosage form of topiramate or salts thereof wherein the composition comprises an enhanced immediate release coated bead population in addition to two extended release (XR) coated bead populations, wherein each XR component comprises a release controlling coating. However, the currently available topiramate compositions suffer from reduced bioavailability i.e., attenuated plasma concentrations due to degradation of the drug in the acidic pH.

Topiramate is also an acid-labile drug. Degradation of topiramate in the acidic environment of the stomach is the likely cause of its reduced bioavailability in the available dosage forms. The current invention addresses this problem by incorporating a specific amount of alkalizer in the coating thus stabilizing the drug in the acidic environment.

Thus, there is still a need for an alternate chemically, physically and organoleptically stable sustained release topiramate composition which could ease and simplify the overall formulation process, and enhance topiramate bioavailability.

The instant invention addresses these and other needs by providing alternate modified compositions of topiramate characterized by a sustained release of topiramate. This invention additionally provides an effective, once-daily dosage form of topiramate or a pharmaceutically acceptable salt thereof, which not only enables an effective single daily dose regimen to improve patient compliance but may also reduce some of the side effects of topiramate compared to the higher daily doses of immediate release topiramate composition. Additionally, the composition involves easy processing without the need to form multiple types of beads with the added advantage of stability and no topiramate degradation with reduced alkalizer quantity.

### SUMMARY OF INVENTION

The present invention is directed in part to topiramate pharmaceutical compositions that allow for a sustained release, preferably once-daily administration that releases topiramate over an extended period of time. The dosage form is preferably at least equivalent in effectiveness to the conventional immediate release, multiple-dose daily regimen, and provides steady-state blood levels of topiramate over a course of treatment. A once-a-day administration of topiramate is advantageous over multiple-dose administration in terms of patient compliance and reduced adverse events, thus providing better treatment of the conditions for which the topiramate is indicated.

In one general aspect, the invention provides a sustained release topiramate composition comprising a single population of beads, wherein each bead comprises:

a) an inert core,

b) an inner layer comprising topiramate,

c) an outer layer comprising at least one sustained release material and at least one alkalizer, wherein the alkalizer comprises about 1 to about 60% by weight of the outer layer.

In another general aspect, there is provided a sustained release topiramate composition, wherein the inner layer forms about 30 to about 45% by weight of the composition.

In another general aspect, there is provided a sustained release topiramate composition, wherein the inert core material is selected from cellulose spheres, silicon dioxide, starch or sugar spheres.

In another general aspect, there is provided a sustained release topiramate composition, wherein the inert core comprises from about 20 to about 60% by weight of the composition.

In another general aspect, there is provided a sustained release topiramate composition, wherein topiramate is present in an amount from 0.5 to 1000 mg.

In another general aspect, there is provided a sustained release topiramate composition, wherein the outer layer comprises an inner barrier layer and an outer sustained release layer.

In yet another general aspect, there is provided a sustained release topiramate composition, wherein the outer layer comprising at least one sustained release material comprises a combination of at least one hydrophobic polymer and at least one hydrophilic polymer.

In another general aspect, there is provided a sustained release topiramate composition, wherein the sustained release material comprises hydroxyalkylcelluloses such as hydroxypropyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate or mixtures thereof.

In another general aspect, there is provided a sustained release topiramate composition, wherein the sustained release material comprises a combination of ethyl cellulose and polyvinylpyrrolidone.

In another general aspect, there is provided a sustained release topiramate composition, wherein the composition is free of an immediate release layer comprising topiramate,

Embodiments of the pharmaceutical composition may include one or more of the following features. For example, the pharmaceutical composition may further include one or more pharmaceutically acceptable excipients. The pharmaceutically acceptable excipients may include one or more binders, fillers, disintegrants, alkalizers/stabilizing agent, lubricants, wetting agents, glidants, pore forming agents, plasticizers and the like.

In another general aspect, there is provided a sustained release topiramate composition, wherein the composition is in the form of a tablet, a pill, a capsule, a caplet, a pouch, sprinkles, beads, granules, or powder.

In another general aspect, there is provided a sustained release topiramate composition, wherein the composition is in the form of a capsule.

In another general aspect, there is provided a sustained release topiramate composition, wherein the composition is in the form of a capsule wherein capsule shell comprises hydroxypropyl methylcellulose with a content of methoxyl groups and hydroxypropoxyl groups combined of 23 to 39% by weight of the hydroxypropyl methyl cellulose.

In another general aspect, there is provided a sustained release topiramate composition, wherein the composition exhibits no significant difference in both rate and extent of absorption of topiramate as compared to extended release formulation of topiramate marketed under the trade name Qudexy XR®.

In yet another general aspect, there is provided a sustained release topiramate composition, wherein the composition provides a mean AUC and a mean Cₘₐₓ of plasma topiramate in both fed and fasted states within 80% to 125% of mean AUC and mean Cₘₐₓ of plasma topiramate provided by a topiramate extended release reference standard upon single dose administration to a population of human subjects respectively.

In yet another general aspect, there is provided a process of preparing the sustained release topiramate composition, wherein the process comprises the steps of:

a) coating an inner layer comprising topiramate on the inert cores,

b) coating the product of step ‘a’ with an outer layer comprising at least one sustained release material and at least one alkalizer to obtain beads,

c) lubricating the beads; and

d) filling the beads into a capsule shell.

In yet another general aspect, there is provided a process of preparing the sustained release topiramate composition, wherein the process comprises the steps of:

a) coating an inner layer comprising topiramate on the inert cores,

b) coating the product of step ‘a’ with a barrier layer comprising at least one alkalizer,

c) coating the product of step ‘b’ with a sustained release layer comprising at least one sustained release material to obtain beads,

d) lubricating the beads; and

e) filling the beads into a capsule shell.

In another general aspect, there is provided a sustained release topiramate composition, wherein the composition releases at least about 90% of topiramate in less than or equal to 8 hours when measured in USP type I (basket) apparatus rotating at 100 rpm using 900 ml of tris buffer having pH 7.5.

Embodiments of the pharmaceutical composition may include one or more of the following features. For example, the pharmaceutical composition may further include one or more pharmaceutically acceptable excipients. The pharmaceutically acceptable excipients may include one or more binders, fillers, disintegrants, alkalizers stabilizing agent, lubricants, wetting agents, glidants, pore forming agents, plasticizers and the like.

The details of one or more embodiments of the invention are set forth in the description below. Other features of the invention will be apparent from the description.

### DETAILED DESCRIPTION OF THE INVENTION

The present invention provides a sustained release topiramate composition for the treatment or prevention of a pathological condition in a mammalian subject wherein topiramate is released from the composition at a sustained rate along a pre-determined release profile. Such release is achieved by incorporation into the composition beads comprising multi-layered inert cores comprising an inner layer and an outer layer, wherein the outer layer comprises at least one alkalizer.

The relative amount of each component in the topiramate composition of the present invention is determined according to the purpose of administration and a pre-determined release profile, and the total amount of topiramate in the composition varies from 0.5 mg to about 1000 mg, preferably selected from 25 mg, 50 mg, 100 mg, 150 mg and 200 mg. In other words, topiramate or a pharmaceutically acceptable salt is present in the composition in an amount of from about 0.5% to about 85% by weight, and preferably of from about 20% to about 70% by weight, even more preferably from about 20% to about 50% by weight of the composition.

The term “about” has been recited here and throughout the specification to account for variations, which can arise from inaccuracies in measurement inherent and understood by those of ordinary skill in the chemical and pharmaceutical arts.

The term “sustained release” as used herein can be used synonymously with extended release, controlled release, modified release and delayed release. The term “sustained release” is defined herein as release of a pharmaceutical agent in a continuous manner over a prolonged period of time.

By “prolonged period of time”, it is meant a continuous period of time of greater than about 1 hour, preferably, greater than about 4 hours, more preferably, greater than about 8 hours, more preferably greater than about 12 hours, more preferably still, greater than about 16 hours up to more than about 24 hours.

The term “bioavailability” refers to an extent to which (and sometimes rate at which) the active moiety (drug or metabolite) enters systemic circulation, thereby gaining access to the site of action.

“AUC” is the area under the plasma concentration-time curve and is considered to be the most reliable measure of bioavailability. It is directly proportional to the total amount of unchanged drug that reaches the systemic circulation.

As used herein, unless otherwise noted, “rate of release” or “release rate” of a drug refers to the quantity of drug released from a dosage form per unit time, e.g., milligrams of drug released per hour (mg/hr) or a percentage of a total drug dose released per hour. Drug release rates for dosage forms are typically measured as an in vitro rate of drug release i.e., a quantity of drug released from the dosage form per unit time measured under appropriate conditions and in a suitable fluid.

The release rates referred to herein are determined by placing a dosage form to be tested in a medium in an appropriate dissolution bath. Aliquots of the medium, collected at pre-set intervals, are then injected into a chromatographic system fitted with an appropriate detector to quantify the amounts of drug released during the testing intervals.

The term “beads”, as used herein, includes, without any limitations on the nature and size thereof, any particles, spheres, granules, pellets or any structural units, preferably coated that may be incorporated into an oral dosage form.

The release rates referred to herein are determined by placing a dosage form to be tested in a medium in an appropriate dissolution bath. Aliquots of the medium, collected at pre-set intervals, are then injected into a chromatographic system fitted with an appropriate detector to quantify the amounts of drug released during the testing intervals.

In another embodiment, the sustained release topiramate composition of the invention comprises an inert core coated by an inner layer comprising topiramate, wherein the inner layer comprises about 20 to about 60% by weight of the composition, more preferably about 25 to about 50% by weight of the composition and even more preferably about 30 to about 45% by weight of the composition.

In another embodiment, the sustained release topiramate composition of the invention comprises at least one alkalizer comprising about 0.1 to about 10% by weight of the composition, more preferably about 0.1 to about 8% by weight of the composition, more preferably about 0.1 to about 5% by weight of the composition, more preferably about 0.1 to about 3% by weight of the composition and even more preferably about 0.1 to about 2% by weight of the composition.

In another embodiment, the sustained release topiramate composition of the invention comprises an inert core coated by an inner layer comprising topiramate, and an outer layer comprising at least one sustained release material and at least one alkalizer, wherein the outer layer comprises about 2 to about 25%, more preferably from about 2 to about 20%, even more preferably from about 5 to about 15% by weight of the composition.

In another embodiment, the sustained release topiramate composition of the invention comprises an inert core coated by an inner layer comprising topiramate and an outer layer comprising at least one alkalizer; wherein the alkalizer comprises about 1 to about 60% by weight of the outer layer, more preferably about 1-50% by weight of the outer layer, even more preferably about 1-25% by weight of the outer layer of the composition.

In yet another embodiment, the sustained release topiramate composition of the invention comprises an inert core coated by an inner layer comprising topiramate and an outer layer, wherein the outer layer further comprises an inner barrier layer and an outer sustained release layer. The inner barrier layer comprises about 1 to about 8%, more preferably from about 2 to about 5% by weight of the outer layer and the outer sustained release layer comprises from about 2 to about 9%, more preferably from about 5 to about 9% by weight of the outer layer.

In another general aspect, there is provided a sustained release topiramate composition, wherein the composition, when subjected to in vitro dissolution testing in USP type II apparatus rotating at 50 rpm using 900 ml of 0.1 N HCl (0-2 hour) followed by 6.8 pH phosphate buffer, releases at least about 20% and not more than about 70% of topiramate within 2 hours in vitro, more preferably at least about 20% and not more than about 60% of topiramate within 2 hours in vitro; at least about 60% and not more than about 80% of topiramate within 4 hours in vitro, more preferably at least about 65% and not more than about 80% of topiramate within 4 hours in vitro; at least about 80% of topiramate within 8 hours in vitro and at least about 90% of topiramate within 10 hours in vitro.

Alternatively, there is provided a sustained release topiramate composition, wherein the composition, when subjected to in vitro dissolution testing in USP type I (basket) apparatus rotating at 100 rpm using 900 ml of tris buffer having pH 7.5, releases at least about 15% and not more than about 35% of topiramate within 1 hour in vitro; at least about 35% and not more than about 65% of topiramate within 2 hours in vitro; at least about 65% and not more than about 90% of topiramate within 4 hours in vitro; and at least about 90% of topiramate within 8 hours in vitro.

In another embodiment, the sustained release topiramate composition of the invention comprises an inert core coated by an inner layer comprising topiramate and an outer layer, wherein the outer layer further comprises an inner barrier layer and an outer sustained release layer, wherein at least one of the inner or outer layers comprise at least one alkalizer.

In another embodiment, the sustained release topiramate composition of the invention comprises an inert core coated by an inner layer comprising topiramate and an outer layer comprising at least one alkalizer and at least one sustained release material. Any suitable sustained release material or suitable combination of sustained release materials may be used. Such materials are known to those skilled in the art. The term “sustained release material” as used herein includes, for example, hydrophilic polymers, hydrophobic polymers and mixtures thereof which are capable of modifying the release of an active ingredient either via coating or in matrix in vitro or in vivo.

Furthermore, in certain embodiments, the capsules of the present disclosure do not include an immediate release component comprising topiramate in any significant amount. Typically, capsules of the present invention are free from an immediate-release component.

The beads of the present invention may further comprise other pharmaceutically active agents suitable for use in combination with topiramate for treatment or prevention of a pathological condition. The additional pharmaceutically active agents, without limitation, may be represented by analgesic and anti-inflammatory compounds such as COX-2 inhibitors, nonsteroidal anti-inflammatory drugs (NSAIDs), narcotic drugs such as opiates and morphinomimetics, synthetic drugs with narcotic properties such as tramadol; anticonvulsants such as valproic acid or its derivatives, carbamazepine, oxcarbazepine, gabapentin, and lamotrigine; anorectics or anti-obesity agents such as sibutramine or other, orlistat or other pancreatic lipase inhibitors, diethylpropion, fluoxetine, bupropion, amphetamine, methamphetamine, sertraline, zonisamide, and metformin, as well as medications associated with weight-gain, such as sulfonylurea derivatives, insulin, and thiazolidinediones whose weight-gain effect is tempered by topiramate; anti-hypertensive agents such as diuretics, anti-adrenergics, calcium channel blockers, ACE inhibitors, angiotensin II receptor antagonists, aldosterone antagonists, vasodilators, centrally acting adrenergic drugs, and adrenergic neuron blockers; mood stabilizers such as various forms/salts of lithium, Omega-3 fatty acids and others known in the art, drugs for treatment or prevention of migraines, such as ergot derivatives or triptans, or any other pharmaceutical or nutraceutical ingredient that can be safely and beneficially combined with topiramate.

Accordingly, the pharmaceutical composition of the invention can include drugs which are chemically unstable in acidic environments but which also have narrow “absorption windows” high up in the GI tract. The pharmaceutical composition of the invention gives good protection in very acidic environments (pH <3) whilst not delaying the release in regions of good drug absorption (pH >4), whether this be the upper intestine or the stomach.

The inert core material useful in the present invention may be selected from, but are not limited to, a group consisting of cellulose spheres, silicon dioxide, starch or sugar spheres. The inert core is present in an amount of from about 10% to about 70% by weight, and preferably in an amount of about 20% to about 60% by weight, even more preferably about 30 to about 60% by weight of the composition.

The sustained release outer layer comprises a sustained release material, and optionally, pore former and other excipients. The sustained release material is preferably selected from a group comprising polymers, such as hydroxyalkylcelluloses such as hydroxypropyl methylcellulose and hydroxypropylcellulose, polyethylene oxide, alkylcelluloses such as methylcellulose and ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyvinyl acetate, acrylic polymers such as polyacrylates, polymethacrylates and copolymers thereof, and other water-based or solvent-based coating materials; and fatty acid alcohols, fatty acids, fatty acid esters, fatty acid glycerides, waxes, hydrogenated castor oil, hydrogenated vegetable oils; and mixture thereof.

One or more sustained release material can be present in an amount of about 0.5% to about 30% by weight, about 0.5% to about 25% by weight, preferably in an amount of about 0.5% to about 20% by weight, even more preferably about 0.5% to about 10% by weight of the composition.

The binder may be present in the composition in an amount of from about 0.1% to about 15% by weight, and preferably from about 0.2% to about 10% by weight of the composition. Suitable binders may include one or more of hydroxyethyl cellulose, hydroxypropyl cellulose, hydroxypropyl methylcellulose, carbomers, dextrin, ethyl cellulose, methylcellulose, shellac, zein, gelatin, polymethacrylates, polyvinyl pyrrolidone, pregelatinized starch, sodium alginate, gums, synthetic resins and the like.

Suitable fillers may include one or more of microcrystalline cellulose, starch, dibasic calcium phosphate, tribasic calcium phosphate, calcium carbonate, dextrose, kaolin, magnesium carbonate, magnesium oxide; sugars such as lactose or sucrose; sugar alcohols such as mannitol, sorbitol, erythritol and the like.

Suitable disintegrants may include one or more of croscarmellose sodium, sodium starch glycolate, pregelatinized starch, sodium carboxymethyl cellulose, cross-linked polyvinylpyrrolidone and the like.

Alkalizers/stabilizing agents may be selected from one or more agents selected from salt of a Group IA metal, an alkali earth metal buffering agent, a calcium buffering agent, a magnesium buffering agent, and an aluminum buffering, sodium bicarbonate, potassium bicarbonate, magnesium hydroxide, magnesium lactate, magnesium gluconate, magnesium oxide, magnesium aluminate, magnesium carbonate, magnesium silicate, magnesium citrate, aluminum hydroxide, aluminum phosphate, aluminum hydroxide/magnesium carbonate, potassium carbonate, potassium citrate, aluminum hydroxide/sodium bicarbonate coprecipitate, aluminum glycinate, aluminum magnesium hydroxide, sodium citrate, sodium tartrate, sodium acetate, sodium carbonate, sodium polyphosphate, sodium dihydrogen phosphate, potassium polyphosphate, sodium pyrophosphate, potassium pyrophosphate, disodium hydrogenphosphate, dipotassium hydrogenphosphate, trisodium phosphate, tripotassium phosphate, potassium metaphosphate, calcium acetate, calcium glycerophosphate, calcium chloride, calcium hydroxide, calcium lactate, calcium carbonate, calcium gluconate, calcium bicarbonate, calcium citrate, calcium phosphate magnesium phosphate, potassium phosphate, sodium phosphate, trihydroxymethylaminomethane, an amino acid, an acid salt of an amino acid, an alkali salt of an amino acid, and combinations of any of the foregoing.

Suitable lubricants and glidants may include one or more of talc, metallic stearates such as magnesium stearate, calcium stearate, zinc stearate; colloidal silicon dioxide, finely divided silicon dioxide, stearic acid, hydrogenated vegetable oil, glyceryl palmitostearate, glyceryl monostearate, glyceryl behenate, polyethylene glycols, powdered cellulose, starch, sodium stearyl fumarate, sodium benzoate, mineral oil, magnesium trisilicate, kaolin; and the like.

Suitable pore forming agents may include glucose, fructose, mannitol, mannose, galactose, sorbitol, pullulan, dextran, hydroxyalkylcelluloses, carboxyalkylcelluloses, hydroxypropylmethylcellulose, cellulose ethers, acrylic resins, polyvinylpyrrolidone, cross-linked polyvinylpyrrolidone, polyethylene oxide, carbomer, diols, polyols, polyhydric alcohols, polyalkylene glycols, polyethylene glycols, polypropylene glycols or block polymers thereof, polyglycols, poly(α, Ω)alkylenediols; alkali metal salts and alkaline earth metal salts, and combinations thereof.

Suitable wetting agents may include non-ionic surfactants, ionic surfactants and combinations thereof.

Suitable plasticizers useful for the purposes of the present invention include such as triethyl citrate, acetyl triethyl citrate, acetyl tributyl citrate, acetylated monoglycerides, glycerin, triacetin, polyethylene glycol, propylene glycol, phthalate esters, castor oil, sorbitol and dibutyl sebacate; anti-tackiness agents like talc and glyceryl monostearate; pigments like titanium dioxide or ferric oxides, polishing agents like glyceryl monostearate, carnauba wax, candellila wax and the like.

The assisting solvents useful for the formulation process of the present invention may be selected from those solvents that can disperse, dissolve, partially dissolve or those that can induce or assist the coalescence or molecular relaxation of the components of the coating layer. Examples of these solvents include but are not limited to organic solvents, such as alcohols, ketones, ethers, esters, amides, amines, hydrocarbons including substituted hydrocarbons such as chlorinated hydrocarbons and aromatic hydrocarbons, furans, sulfoxides, organic acids, phenols, super-critical fluids; ammonia; and water, buffered water, or water solutions of other inorganic or organic compounds, and their combinations.

The compositions may be prepared using processes known to one skilled in the art.

In one embodiment, the compositions may be prepared by coating topiramate dispersed/dissolved with one or more pharmaceutically acceptable excipients in a solvent on inert cores forming an inner layer. The resultant coated cores may be coated with an outer layer comprising at least one alkalizer and at least one sustained release material.

The coating may be seal coating, film coating, subcoating, barrier coating, polishing coating, compression coating, fast disintegrating coating, enzyme degradable coating, sugar coating, release-modifying coating like polymeric or enteric coat, specialized coatings like bioadhesive coatings, and such the like, or combinations thereof. Coating may be achieved by methods such as by using fluidized bed equipment, bottom-spray fluid-bed coating (e.g., Wurster), top-spray fluid-bed coating, and tangential-spray fluid-bed coating, perforated pans, a regular pharmaceutical pan, compression coating, continuous or short spray methods or such other methods known to one skilled in the art. Further, multiple coatings may be applied to achieve desired results. The inert cores may be coated with active substance along with pharmaceutically acceptable excipient(s) and may be further coated with sustained release or specialized coating. There may be a separate coating of alkalizer or it may be present in any of the coating layers. The coatings may further comprise other excipients like plasticizers.

In some embodiments, it may be further desirable to optionally coat the beads with an outer layer comprising an “inner barrier layer” to provide protection against topiramate degradation and an “outer sustained release” layer to provide a sustained release mechanism to the composition. The beads/final composition may further be coated with an “overcoat” to provide, e.g., moisture protection, static charge reduction, taste-masking, flavoring, coloring, and/or polish or other cosmetic appeal. Suitable coating materials for such an overcoat are known in the art, and include, but are not limited to, cellulosic polymers such as hydroxypropylmethylcellulose, hydroxypropylcellulose and microcrystalline cellulose, or combinations thereof (for example various Opadry® coating materials).

Topiramate is introduced to the inert cores by techniques known to one skilled in the art, such as drug layering, powder coating, extrusion/spheronization, roller compaction or granulation. Preferably, the introduction method is drug layering by spraying a suspension of topiramate and one or more pharmaceutically acceptable excipients onto the inert cores.

In another embodiment, the compositions may be prepared by dissolving polyvinylpyrrolidone and polyethylene glycol in purified water followed by topiramate addition to form dispersion. The obtained topiramate dispersion may then be coated on to the inert cores using fluid bed equipment to obtain beads. After topiramate loading on the inert core particles, the obtained beads may be dried, followed by application of a barrier layer with opadry solution and at least one alkalizer to form double coated beads. Further, the sustained release coating dispersion comprising ethyl cellulose and polyvinylpyrrolidone may sprayed onto the double coated bead bed to evenly coat to a desired coating level. The beads may further be lubricated using talc.

In another embodiment, the compositions may be prepared by dissolving polyvinylpyrrolidone and polyethylene glycol in purified water followed by topiramate addition to form dispersion. The obtained topiramate dispersion may then be coated on to the inert cores using fluid bed equipment to obtain beads. After topiramate loading on the inert core particles, the obtained beads may be dried, followed by application of a barrier layer with opadry solution to form double coated beads. Further, the sustained release coating dispersion comprising ethyl cellulose, polyvinylpyrrolidone and at least one alkalizer e.g., sodium carbonate anhydrous may be sprayed onto the double coated bead bed to evenly coat to a desired coating level. The beads may further be lubricated using talc.

In another embodiment, the compositions may be prepared by dissolving polyvinylpyrrolidone and polyethylene glycol in purified water followed by topiramate addition to form dispersion. The obtained topiramate dispersion may then be coated on to the inert cores using fluid bed equipment to obtain beads. These beads may further be spray coated with an outer layer comprising ethyl cellulose and polyvinylpyrrolidone and at least one alkalizer e.g. sodium bicarbonate. The beads may further be lubricated using talc.

An amount of beads sufficient to deliver the desired dose may be for example compressed into a tablet, filled into pouches, encapsulated into a capsule of any desirable size, for example, a size 000, 00, 0el, 0, 1, 2, 3, 4, or 5.

Components of a suitable capsule shell include, but are not limited to, hydroxypropyl methylcellulose and gelatin. Preferably, a capsule shell is a hydroxypropyl methylcellulose (HPMC) shell. Typically, commercially available HPMC capsules include small amounts of water, colorants (e.g., TiO₂ and iron oxides), and optionally gelling agents and gelling promoters. They have relatively low moisture content, making them suitable for moisture-sensitive materials. Such capsules resist breakage even at low moisture levels. However, the capsules of cellulose ether film suffer from the problem that the gelling aid which is blended for assisting in film formation will precipitate out on the film surface during long-term storage. During long-term storage of these cellulose ether film capsules, the water content of the film can be lowered owing to the storage environment or the water absorption of the fill. Then the potassium or calcium ions as the gelling aid re-form potassium chloride or calcium chloride which precipitates out on the film surface. U.S. Pat. No. 6,649,180 suggests means to overcome this problem by limiting the total content of alkoxyl and hydroxyalkoxyl groups in the cellulose ether to 37.6% by weight. More particularly, the total content corresponds to the total content of methoxyl groups (abbreviated as “MO groups”) and hydroxypropoxyl groups (abbreviated as “HPO groups”) in the case of HPMC is considered. However, surprisingly the compositions of the present invention were found to be physically and organoleptically stable during the entire stability conditions i.e. the capsule shells showed no signs of white precipitates. HPMC capsules shells used for the present invention preferably comprise methoxyl groups and hydroxypropoxyl groups combined of 23 to 39% by weight of the hydroxypropyl methyl cellulose; more preferably 30 to 39%, more preferably 35 to 39%, even more preferably 37 to 39% and still more preferably 37.6 to 39%.

In a general aspect, there is provided a method of treatment of a neurological and/or psychiatric condition, comprising orally administering to the subject a therapeutically effective amount of the sustained release composition of the invention, wherein said condition is selected from a group consisting of epilepsy, migraine, essential tremor, restless limb syndrome, cluster headaches, neuralgia, neuropathic pain, Tourette's syndrome, infantile spasms, bipolar disorder, dementia, depression, psychosis, mania, anxiety, schizophrenia, obsessive-compulsive disorder, post-traumatic stress disorder, attention deficit hyperactivity disorder, impulse control disorders, border line personality disorder, addiction, autism, chronic neurodegenerative disorders, acute neurodegeneration, amyotrophic lateral sclerosis.

In one embodiment, the compositions of the present invention are found to be stable and may retain at least 90% of the potency of topiramate in the composition after storing the composition at 40° C. and 75% relative humidity for at least three months, preferably at least six months.

In yet another embodiment, the sustained release composition of topiramate s exhibits no significant difference in both rate and extent of absorption of topiramate as compared to extended release capsule composition of topiramate marketed under the trade name Qudexy XR®.

In yet another embodiment, the sustained release compositions provide for a mean Cₘₐₓ in the range of 80% to 125%, as compared to the currently marketed topiramate extended release capsule formulation Qudexy XR®.

In yet another embodiment, the sustained release compositions provide for a mean AUC in the range of 80% to 125%, as compared to the currently marketed topiramate extended release capsule formulation Qudexy XR®.

The invention is further illustrated by the following examples which are provided to be exemplary of the invention and do not limit the scope of the invention. While the present invention has been described in terms of its specific embodiments, certain modifications and equivalents will be apparent to those skilled in the art and are intended to be included within the scope of the present invention.

### EXAMPLE 1

TABLE 1

| | | |
|---|---|---|
| Sr. | | |
| No. | Ingredient | % w/w |

Inner coating

| | | |
|---|---|---|
| 1. | Microcrystalline cellulose | 54.84 |
| 2. | Topiramate | 32.68 |
| 3. | Polyvinylpyrrolidone (K 90) | 1.96 |
| 4. | Polyethylene glycol (400 NF) | 0.39 |
| 5. | Purified water | q.s |

Barrier coating

| | | |
|---|---|---|
| 6. | Opadry clear | 1.34 |
| 7. | Sodium carbonate anhydrous | 0.82 |
| 8. | Talc (micronized) | 0.54 |
| 9. | Purified water | q.s |

Polymer coating

| | | |
|---|---|---|
| 10. | Ethyl cellulose (10 cps) | 3.93 |
| 11. | Polyvinylpyrrolidone (K 30) | 1.85 |
| 12. | Triethyl citrate | 0.58 |
| 13. | Talc | 0.58 |
| 14. | Methylene chloride | q.s |
| 15. | Isopropyl alcohol | q.s |

Lubrication

| | | |
|---|---|---|
| 16. | Talc | 0.49 |

Process:
a) Polyvinylpyrrolidone and Polyethylene glycol were dissolved in water until a clear solution was obtained,
b) topiramate was dispersed into the step-a solution,
c) microcrystalline cellulose inert cores were loaded in a fluid bed equipment,
d) dispersion obtained in step-b) was coated onto the inert cores to obtain beads,
e) opadry clear, sodium carbonate anhydrous and talc were dispersed in purified water and stirred for about 30 to about 60 minutes,
f) the dispersion of step-e) was loaded on the beads of step-d) followed by drying and fractionating,
g) ethyl cellulose, polyvinylpyrrolidone and triethyl citrate were added into a solvent mixture of methylene chloride and isopropyl alcohol and mixed,
h) talc was added to step-g) solution to form a dispersion,
i) beads of step-f) were loaded in a fluid bed equipment and coated with talc dispersion of step-h),
j) beads were dried, fractionated and lubricated with talc,
k) the beads obtained were filled into HPMC capsule shells.

The dissolution performance for the topiramate composition of the invention was measured using a USP dissolution apparatus Type I (Basket) and the amount of drug released was analyzed via UV analysis. Release times were measured by allowing the composition to sink in the dissolution vessel and operating the apparatus at 100 rpm. Aliquots were withdrawn from tris buffer at pH 7.5 up to 8 hours.

TABLE 1a
Dissolution performance for the composition of Example 1 in USP
type I (basket) apparatus rotating at 100 rpm using 900 ml of tris buffer
having pH 7.5.

| Time (Hr) | % drug release |
|---|---|
| 1 | 25.4 |
| 2 | 52.7 |
| 3 | 69.3 |
| 4 | 82.3 |
| 6 | 95.7 |
| 8 | 98.5 |

Bioequivalence Study

In-vivo study was conducted in healthy human volunteers to assess bioavailability of topiramate sustained release capsules (Test—composition of the invention as per Example 1) and Qudexy XR® (Reference—Marketed topiramate extended release capsules).

TABLE 1b
Summary of PK parameters of Reference and Test compositions
under Fasting condition:

| | REFERENCE | TEST GEOMETRIC |
|---|---|---|
| PARAMETER | GEOMETRIC MEANS | MEANS |
| Cₘₐₓ (ng/ml) | 3287.87 | 3407.01 |
| AUCₜ (ng * h/ml) | 178982.59 | 178722.22 |
| AUCᵢ (ng * h/ml) | 183754.20 | 183695.74 |

TABLE 1c
Summary of PK parameters of Reference and Test compositions
under Fed condition:

| | REFERENCE | TEST GEOMETRIC |
|---|---|---|
| PARAMETER | GEOMETRIC MEANS | MEANS |
| Cₘₐₓ (ng/ml) | 3018.21 | 3511.12 |
| AUCₜ (ng * h/ml) | 163959.32 | 171861.07 |
| AUCᵢ (ng * h/ml) | 168807.21 | 177018.70 |

1. A sustained release topiramate composition comprising a single population of beads, wherein each bead comprises:
a) an inert core,
b) an inner layer comprising topiramate,
c) an outer layer comprising at least one sustained release material and at least one alkalizer, wherein the alkalizer comprises about 1 to about 60% by weight of the outer layer.

2. The sustained release topiramate composition according to claim 1, wherein the inner layer forms about 30 to about 45% by weight of the composition.

3. The sustained release topiramate composition according to claim 1, wherein the inert core is selected from cellulose spheres, silicon dioxide, starch or sugar spheres.

4. The sustained release topiramate composition according to claim 1, wherein the inert core comprises from about 20 to about 60% by weight of the composition.

5. The sustained release topiramate composition according to claim 1, wherein topiramate is present in an amount from 0.5 to 1000 mg.

6. The sustained release topiramate composition according to claim 1, wherein the outer layer comprises an inner barrier layer and an outer sustained release layer.

7. The sustained release topiramate composition according to claim 1, wherein the outer layer comprising at least one sustained release material comprises a combination of at least one hydrophobic and at least one hydrophilic polymer.

8. The sustained release topiramate composition according to claim 1, wherein the sustained release material comprises hydroxypropyl methylcellulose, hydroxypropylcellulose, hydroxyethylcellulose, polyethylene oxide, methylcellulose, ethylcellulose, polyethylene glycol, polyvinylpyrrolidone, cellulose acetate, cellulose acetate butyrate, cellulose acetate phthalate, cellulose acetate trimellitate, polyvinylacetate phthalate, polyalkylmethacrylates, polyvinyl acetate or combinations thereof.

9. The sustained release topiramate composition according to claim 1, wherein the sustained release material comprises a combination of ethyl cellulose and polyvinylpyrrolidone.

10. The sustained release topiramate composition according to claim 1, wherein the composition is free of an immediate release layer.

11. The sustained release topiramate composition according to claim further comprises one or more pharmaceutically acceptable excipients comprising fillers, binders, disintegrants, alkalizers/stabilizing agents, lubricants, wetting agents, glidants, pore forming agents and plasticizers.

12. The sustained release topiramate composition according to claim 1, wherein the composition is in the form of a tablet, a pill, a capsule, a caplet, a pouch, sprinkles, beads or granules.

13. The sustained release topiramate composition according to claim 1, wherein the composition is in the form of a capsule.

14. The sustained release topiramate composition according to claim 1, wherein the composition is in the form of a capsule and wherein a shell of the capsule comprises hydroxypropyl methylcellulose with a content of methoxyl groups and hydroxypropoxyl groups combined of 23 to 39% by weight of the hydroxypropyl methylcellulose.

15. The sustained release composition according to claim 1 is prepared by the process comprising the steps of:
a) coating an inner layer comprising topiramate on the inert cores,
b) coating the product of step ‘a’ with an outer layer comprising at least one sustained release material and at least one alkalizer to obtain beads,
c) lubricating the beads; and
d) filling the beads into a capsule shell.

16. The sustained release composition according to claim 1 is prepared by the process comprising the steps of:
a) coating an inner layer comprising topiramate on the inert cores,
b) coating the product of step ‘a’ with a barrier layer comprising at least one alkalizer,
c) coating the product of step ‘b’ with a sustained release layer comprising at least one sustained release material to obtain beads,
d) lubricating the beads; and
e) filling the beads into a capsule shell.

17. The sustained release topiramate composition according to claim 1, wherein the composition releases at least about 90% of topiramate in less than or equal to 8 hours when measured in USP type I (basket) apparatus rotating at 100 rpm using 900 ml of tris buffer having pH 7.5.

18. The sustained release topiramate composition according to claim 1, wherein the composition releases at least about 15% and not more than about 35% of topiramate within 1 hour in vitro; at least about 35% and not more than about 65% of topiramate within 2 hours in vitro; at least about 65% and not more than about 90% of topiramate within 4 hours in vitro; and at least about 90% of topiramate within 8 hours in vitro when measured in USP type I (basket) apparatus rotating at 100 rpm using 900 ml of tris buffer having pH 7.5.

19. The sustained release topiramate composition according to claim 1, wherein the composition retains at least 90% of the potency of topiramate in the pharmaceutical composition after storage at 40° C. and 75% relative humidity for at least three months.

20. The sustained release topiramate composition according to claim 1, wherein the composition exhibits no significant difference in both rate and extent of absorption of topiramate as compared to extended release capsule formulation of topiramate marketed under the trade name Qudexy XR®.

21. The sustained release topiramate composition according to claim 1, wherein the composition provides a mean AUC of plasma topiramate in both fed and fasted states within 80% to 125% of a mean AUC of plasma topiramate provided by a topiramate extended release reference standard upon single dose administration to a population of human subjects.

22. The sustained release topiramate composition according to claim 1, wherein the composition provides a mean Cₘₐₓ of plasma topiramate in both fed and fasted states within 80% to 125% of a mean Cₘₐₓ of plasma topiramate provided by a topiramate extended release reference standard upon single dose administration to a population of human subjects.
